(19) United States
(12) Patent Application Publication
(10) Pub. No.: US 2025/0223531 A1
(43) Pub. Date: Jul. 10, 2025

(54) BIONIC ORGAN DEVICE

(71) Applicant: DARWIN PRECISIONS CORPORATION, Hsinchu County (TW)

(72) Inventors: Ting-Chieh Yang, Hsinchu County (TW); Pang-Chun Liu, Hsinchu County (TW); Jui-Hua Lin, Hsinchu County (TW); Yu-Hsin Wu, Hsinchu County (TW)

(21) Appl. No.: 19/014,288

(22) Filed: Jan. 9, 2025

(30) Foreign Application Priority Data
Jan. 10, 2024 (TW) 113101087

(51) Int. Cl. C12M 3/00; C12M 1/00; C12M 1/12; C12M 1/42

(52) U.S. Cl. CPC C12M 21/08; C12M 23/20; C12M 25/02; C12M 35/04

(57) ABSTRACT

A bionic organ device includes an organ chip and a temperature control module. The organ chip includes a first body, a second body, and a temperature-sensitive film. The temperature-sensitive film is disposed between the first body and the second body, contains hydrophilic polymer material, and forms a flow channel system with the first body and the second body, where the flow channel system includes a first passage and a second passage. The first passage is located between the first body and the temperature-sensitive film, and the second passage is located between the second body and the temperature-sensitive film. The temperature control module includes a thermally conductive material layer and a temperature controller, and the thermally conductive material layer and the hydrophilic polymer material form the temperature-sensitive film. The temperature controller is connected to the thermally conductive material layer and adjusts the temperature of the thermally conductive material layer.

### FIELD OF THE INVENTION

The present invention relates to a bionic technology, and in particular to a bionic organ device capable of simulating a micro-environment within an organism.

### BACKGROUND OF THE INVENTION

Conventional cell culture models cannot reflect the complex physiological functions of tissues and organs of organisms, while animal experiments have disadvantages such as long cycles and high costs. Moreover, it is always difficult to directly predict the real reactions of organisms. The organ chip mimics key functions of an organism organ, reconstructs a physiological environment of an organ in vivo, simulates a structure, a micro-environment, and physiological functions of the organism organ, and accurately controls parameters. In addition, the organ chip has advantages such as miniaturization, integration, high efficiency, and reduced costs. Furthermore, to simulate the stretching and shrinking of organ cells, the existing organ chip is provided with a vacuum system, which performs vacuum suction to stretch cells, thereby achieving a bionic effect. However, vacuum stretching also pulls a membrane to which cells attach, causing membrane damage and an organ chip malfunction. The manufacturing process of the vacuum system is complex and therefore requires improvement.

### SUMMARY OF THE INVENTION

The present invention provides a bionic organ device, which can be used to simulate a dynamic micro-environment of an organ and has a simplified structure, conducive to simplifying the manufacturing process, reducing the costs, and improving the yield.

The bionic organ device provided by the present invention includes an organ chip and a temperature control module. The organ chip includes a first body, a second body, and a temperature-sensitive film. The temperature-sensitive film is disposed between the first body and the second body, contains a hydrophilic polymer material, and forms a flow channel system with the first body and the second body. The flow channel system includes a first passage and a second passage. The first passage is located between the first body and the temperature-sensitive film, and the second passage is located between the second body and the temperature-sensitive film. The temperature control module includes a thermally conductive material layer and a temperature controller, and the thermally conductive material layer and the hydrophilic polymer material form the temperature-sensitive film. The temperature controller is connected to the thermally conductive material layer and is used to adjust the temperature of the thermally conductive material layer.

With the use of the temperature control module and the temperature-sensitive film, the organ chip of the present invention can be used to simulate the stretching or shrinking of organs, tissues, or cells, and is convenient during use. Moreover, the organ chip of the present invention has a simplified structure and thus is conducive to simplifying the manufacturing process, reducing the costs, and improving the yield.

### BRIEF DESCRIPTION OF THE DRAWINGS

FIG. 1 is a schematic three-dimensional diagram of a bionic organ device according to an embodiment of the present invention;

FIG. 2 is a partially schematic three-dimensional exploded view of a bionic organ device according to an embodiment of the present invention;

FIG. 3 is a schematic cross-sectional view of the bionic organ device, taken along the line A-A in FIG. 1;

FIG. 4 is a schematic top view of a temperature-sensitive film according to an embodiment of the present invention;

FIG. 5 is a schematic top view of a thermally conductive material layer according to an embodiment of the present invention; and

FIG. 6 is a schematic cross-sectional view of the temperature-sensitive film, taken along the line B-B in FIG. 4.

### DETAILED DESCRIPTION OF PREFERRED EMBODIMENTS

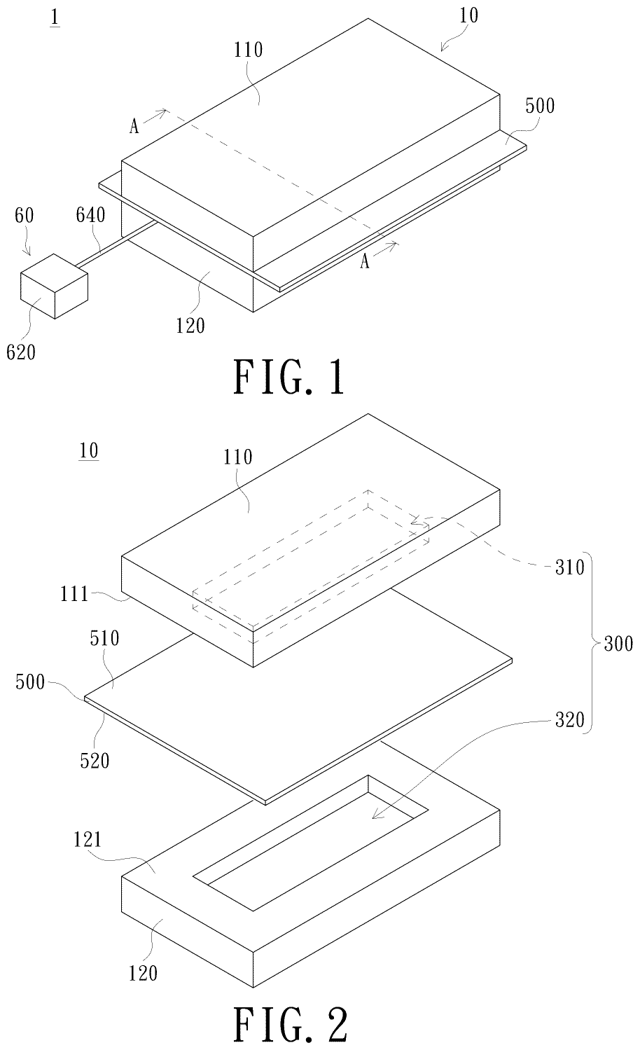

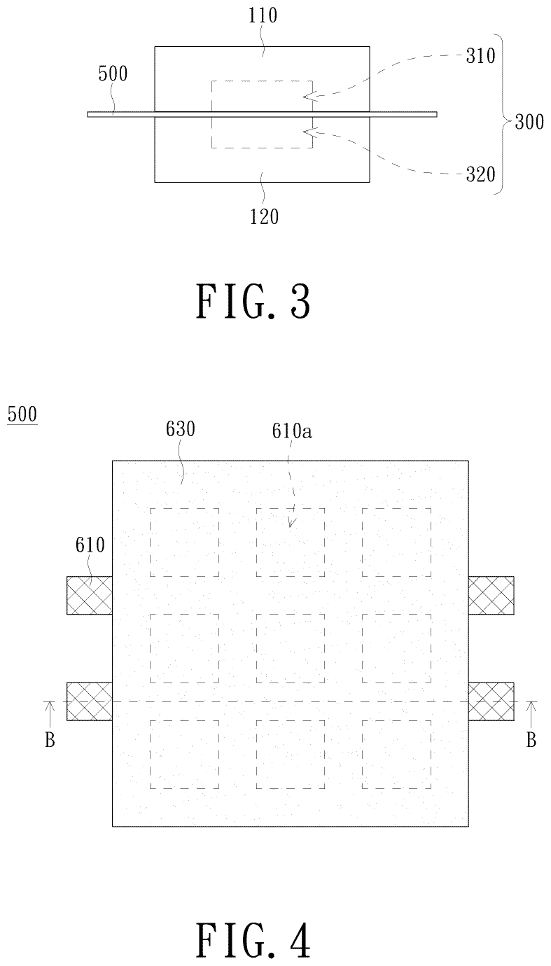

FIG. 1 is a schematic three-dimensional diagram of a bionic organ device according to an embodiment of the present invention, FIG. 2 is a partially schematic three-dimensional exploded view of a bionic organ device according to an embodiment of the present invention, and FIG. 3 is a schematic cross-sectional view of the bionic organ device, taken along the line A-A in FIG. 1. As shown in FIGS. 1 to 3, the bionic organ device of the embodiments of the present invention includes an organ chip 10 and a temperature control module 60, and the temperature control module 60 is connected to the organ chip 10. The organ chip 10 includes a first body 110, a second body 120, and a temperature-sensitive film 500, where the temperature-sensitive film 500 is disposed between the first body 110 and the second body 120. Specifically, the first body 110 and the second body 120 may have a groove-shaped structure, which has an accommodating space and an opening. And the opening of the first body 110 is opposite the opening of the second body 120. They and the temperature-sensitive film 500 form the main parts of the organ chip 10.

As shown in FIGS. 2 and 3, the temperature-sensitive film 500 may be connected to a surface 111 of the first body 110 facing the second body 120 and a surface 121 of the second body 120 facing the first body 110. The connections therebetween can be achieved using one or more known methods such as hot pressing, melting, bonding, or another achievable method. The temperature-sensitive film 500 forms a flow channel system 300 with the first body 110 and the second body 120. The flow channel system includes a first passage 310 located between the first body 110 and the temperature-sensitive film 500 and a second passage 320 located between the second body 120 and the temperature-sensitive film 500. The flow channel system 300 may further include an input/injection pore (not shown in the figure), which can communicate with the first passage 310 and/or the second passage 320. The input/injection pore can be provided in the organ chip 10 by any known means. For example, it can be formed in the first body 110 and/or the second body 120, further enabling communication between the inside and the outside of the organ chip 10.

The first passage 310 and/or the second passage 320 may be used for at least one fluid to pass through or stay therein, and two opposite sides of the temperature-sensitive film 500 have surfaces. A first film surface 510 is located in the first passage 310, a second film surface 520 is located in the second passage 320, and the fluid may be in contact with the first film surface 510 and the second film surface 520 and may further cover them. The temperature-sensitive film 500 reacts when the temperature changes and can preferably expand or shrink due to a temperature change. For example, the temperature-sensitive film 500 expands when the temperature rises, and the temperature-sensitive film 500 shrinks when the temperature decreases. In the embodiments of the present invention, the temperature-sensitive film 500 includes a hydrophilic polymer material 630.

The temperature-sensitive film 500 may serve as a cell attachment membrane for cells to attach to the first film surface 510 and/or the second film surface 520 to be further cultured in the foregoing fluid. The same or different cells can attach to the first film surface 510 and the second film surface 520, and the fluids in the first passage 310 and the second passage 320 can vary based on the types of the cells. For example, in several embodiments of the present invention, the first film surface 510 is used for the attachment of alveolar epithelial cells, and the second film surface 520 is used for the attachment of microvascular endothelial cells. Accordingly, the first passage 310 is supplied with an oxygen-containing gas, while the second passage 320 is supplied with a culture solution.

The temperature control module 60 includes a thermally conductive material layer 610 and a temperature controller 620. The thermally conductive material layer 610 is disposed in the organ chip 10 and forms the temperature-sensitive film 500 with the hydrophilic polymer material 630. The temperature controller 620 is connected to the thermally conductive material layer 610 and can adjust the temperature of the thermally conductive material layer 610. In the embodiments of the present invention, the temperature controller 620 is located outside the organ chip 10 and may be connected to the thermally conductive material layer 610 via, for example, a wire rod. For example, the temperature controller 620 is connected to the thermally conductive material layer 610 via a wire. The thermally conductive material of the thermally conductive material layer 610 may be metal, alloy, metal compound, or non-metal, and is preferably a material with a high thermal conductivity. For example, preferably, the thermal conductivity of the thermally conductive material is not less than 200 W/m·K. Metal of the thermally conductive material may specifically be gold, silver, copper, aluminum, or the like. The temperature controller 620 may be provided in any known manner of controlling temperature or based on the change of the known manner. The temperature controller 620 in the embodiments of the present invention may be, for example, an ON/OFF temperature controller, a PID temperature controller, a FUZZY temperature controller, a combination thereof, or in other types. The present invention does not limit the types of the temperature controller 620; that is, any temperature controller belongs to the scope of the present invention as long as it can adjust the temperature of the thermally conductive material layer 610 within a temperature range (as explained later).

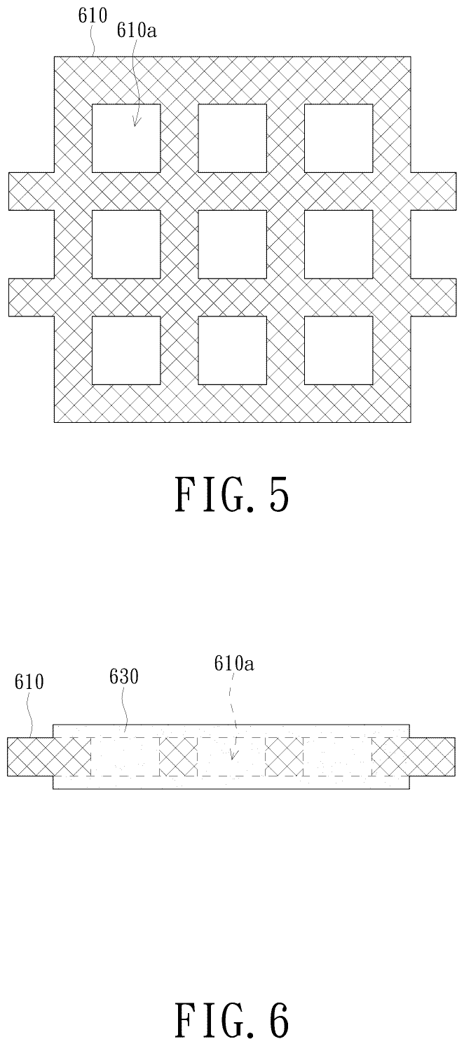

In the embodiments of the present invention, preferably, the thermally conductive material layer 610 is a porous structure and the hydrophilic polymer material 630 can fill a plurality of pores of the thermally conductive material layer 610. The thermally conductive material layer 610 is typically more rigid than the hydrophilic polymer material 630 and can serve as a support structure of the hydrophilic polymer material 630, maintaining the shape of the temperature-sensitive film 500 and enhancing the strength of the temperature-sensitive film 500. In several embodiments of the present invention, the thermally conductive material layer 610 is a metal material layer, which may be, for example, a metal mesh. As shown in FIGS. 4 and 5, the hydrophilic polymer material 630 coats the thermally conductive material layer 610 formed by the metal mesh and fills the pores 610a of the metal mesh. The pores 610a typically have a pore size in a unit of millimeter (mm), for example, a size of 0.03 cm to 0.1 cm, and its quantity is not limited.

The thermally conductive material layer 610 reacts when the temperature changes and can preferably expand or shrink in response to a temperature change. The expansion or shrinking level can vary based on the thermally conductive material. In several embodiments of the present invention, for example, the thermally conductive material may have a linear coefficient of thermal expansion of, for example, 10 to 30 μm/m-° C. When the thermally conductive material layer 610 expands/shrinks, it can cause the hydrophilic polymer material 630 thereon to expand or shrink, such that the temperature-sensitive film 500 expands or shrinks. In addition, the thermally conductive material layer 610 can also provide a thermally conductive function. In another embodiment of the present invention, when the temperature of the thermally conductive material layer 610 changes, the hydrophilic polymer material 630 in contact with it can, for example, expand or shrink in response to the temperature change. During thermal conduction, the level of temperature change of the hydrophilic polymer material 630 may be the same as or different from that of the thermally conductive material layer 610. For example, the thermal conductivity of the hydrophilic polymer material 630 may be less than that of the thermally conductive material. In summary, the thermally conductive material layer 610 can enable the expansion or shrinking of the temperature-sensitive film 500 through effects such as expansion/shrinking and thermal conduction.

In the embodiments of the present invention, the hydrophilic polymer material 630 is a biocompatible material, which is flexible, stretchable, breathable, or porous. The pores of the hydrophilic polymer material 630 generally have a pore size in a unit of nanometer (nm) and can cover a range of pore sizes, for example, tens of nanometers, dozens of nanometers, or hundreds of nanometers. Due to the breathability or porosity of the hydrophilic polymer material 630, small molecules on both sides of the temperature-sensitive film 500 have a chance to move between the first passage 310 and the second passage 320 through the temperature-sensitive film 500. The hydrophilic polymer material 630 can contain functional groups such as —OH, —CONH, —CONH₂, —COOH, —SO₃H, and can be formed by chemically or physically crosslinking monomers that possess these functional groups. In the embodiments of the present invention, the hydrophilic polymer material 630 may be a natural material or a synthetic material. The natural material includes a polysaccharide such as cellulose, starch, hyaluronic acid, alginate, chitosan, or a polypeptide such as collagen, poly-L-lysine, poly-L-glutamic acid, but is not limited thereto. The synthetic material may be, for example, an artificial synthetic polymer such as polyacrylic acid, polymethacrylic acid, or polyacrylamide.

In the embodiments of the present invention, the hydrophilic polymer material 630 is a hydrogel. The hydrophilic polymer material 630 is a hydrogel, which may be obtained by crosslinking, for example, acrylic acid or its derivative, acrylamide or its derivative, and hydroxyethyl methacrylate or its derivative, but is not limited thereto. The hydrogel may be formed in a thermosetting or thermoplastic manner. For example, a hydrogel in a sol or fluid state can be formed by heating or cooling in a mold. The hydrogel in the sol state can coat the thermally conductive material layer 610 and then be heated or cooled to form the temperature-sensitive film 500. In several embodiments of the present invention, the thermally conductive material layer 610 may serve as a substrate, and the hydrogel is grafted onto the substrate of the thermally conductive material layer 610 to form the temperature-sensitive film 500.

Through the use of the temperature controller 620, the temperature of the thermally conductive material layer 610 can change in a range of 25° C. to 65° C., but is not limited thereto. This range can change due to, for example, a different thermally conductive material. For example, when the thermally conductive material is sensitive to temperature, the change range of the temperature is small, for example, a range less than 25° C. to 65° C. In response to, for example, the different patterns of the thermally conductive material layer 610, the expansion coefficient of the thermally conductive material, the thermal conductivity of the thermally conductive material, the type of the hydrophilic polymer material 630, the expansion coefficient of the hydrophilic polymer material 630, and a contact area between the thermally conductive material layer 610 and the hydrophilic polymer material 630, the thermally conductive material layer 610 can cause the temperature-sensitive film 500 to expand or shrink quickly or slowly. For example, in the case of a large expansion coefficient of the thermally conductive material, the thermally conductive material layer 610 may affect the hydrophilic polymer material 630 to a greater extent, such that the temperature-sensitive film 500 expands or shrinks significantly, but this is not limited thereto. In the embodiments of the present invention, the expansion or shrinking of the temperature-sensitive film 500 can be reflected in the change in length, area, and/or volume. Using the change in length as an example, the temperature-sensitive film 500 typically has an expansion change value and a shrinking change value in units of nanometers. In several embodiments of the present invention, for example, the expansion change value of the temperature-sensitive film 500 may be 1 to 20 μm, preferably 5 to 15 μm, and more preferably 8 to 12 μm; and the shrinking change value may be 1 to 20 μm, and preferably 8 to 20 μm.

As shown in FIGS. 1 and 3, the temperature control module 60 further includes a wire set 640 connecting the temperature controller 620 and the thermally conductive material layer 610. As shown in the figure, the wire set 640 can extend from one end of the temperature-sensitive film 500 to be connected to the temperature controller 620, but is not limited thereto. For example, the wire set 640 may further be provided on two adjacent sides, two opposite sides, or each side of the temperature-sensitive film 500. In addition to adjusting the temperature of the thermally conductive material layer 610 in a proper temperature range or expansion/shrinking of the temperature-sensitive film 500, the temperature controller 620 can enable the temperature-sensitive film 500 to have a periodic expansion/shrinking frequency or a specific expansion/shrinking frequency in response to a temperature change and thus can serve as a cell attachment membrane, allowing for, for example, stretching or shrinking of cells in an environment of an organism. The combination use with the flow channel system 300 allows for a more realistic simulation of the dynamic micro-environment of an organ.

With the use of the temperature control module 60 and the temperature-sensitive film 500, the present invention provides a method, for simulating expansion/shrinking of organs, tissues, or cells, completely different from the conventional vacuum method. Expansion/shrinking of organs, tissues, or cells can be simulated by setting the temperature controller 620 outside the organ chip 10 by a user. This, compared with extracting or delivering gas by a vacuum system, is more convenient. In addition, the conventional vacuum system needs to be provided with a passage for extracting and delivering gas in the organ chip, so as to complete simulation. Under comparison, the organ chip 10 of the present invention does not need a vacuum system and therefore has a simplified structure and manufacturing process, which is conducive to reducing costs and improving the yield.

What is claimed is:

1. A bionic organ device, comprising an organ chip and a temperature control module, wherein
the organ chip comprises a first body, a second body, and a temperature-sensitive film, the temperature-sensitive film is disposed between the first body and the second body and has a hydrophilic polymer material, the temperature-sensitive film forms a flow channel system with the first body and the second body, and the flow channel system comprises:
a first passage, located between the first body and the temperature-sensitive film; and
a second passage, located between the second body and the temperature-sensitive film; and
the temperature control module comprises a thermally conductive material layer and a temperature controller, the thermally conductive material layer and the hydrophilic polymer material form the temperature-sensitive film, and the temperature controller is connected to the thermally conductive material layer and is configured to adjust a temperature of the thermally conductive material layer.

2. The bionic organ device according to claim 1, wherein the temperature-sensitive film serves as a cell attachment membrane, the temperature-sensitive film has a first film surface in the first passage and a second film surface in the second passage, and the first film surface, the second film surface, or the combination thereof is used for cell attachment.

3. The bionic organ device according to claim 1, wherein the first passage, the second passage, or the combination thereof is used for at least one fluid to pass therethrough, and the at least one fluid is in contact with at least one surface of the temperature-sensitive film.

4. The bionic organ device according to claim 1, wherein the temperature-sensitive film further comprises a metal mesh, and the hydrophilic polymer material coats the metal mesh and fills pores of the metal mesh.

5. The bionic organ device according to claim 1, wherein a thermally conductive material of the thermally conductive material layer is metal, alloy, metal compound, non-metal, or the combination thereof with a thermal conductivity of not less than 200 W/m·K.

6. The bionic organ device according to claim 1, wherein the thermally conductive material layer is a metal material layer, the hydrophilic polymer material is a hydrogel, and the hydrophilic polymer material coats the metal material layer.

7. The bionic organ device according to claim 1, wherein the thermally conductive material layer is suitable for expanding or shrinking in response to temperature to expand or shrink the temperature-sensitive film.

8. The bionic organ device according to claim 7, wherein the temperature controller is further connected to the thermally conductive material layer via a wire and configured to adjust a temperature of the thermally conductive material layer in a temperature range.

9. The bionic organ device according to claim 8, wherein the temperature range is 25° C. to 65° C., and the temperature-sensitive film is suitable for expansion or shrinking in the temperature range and for a length change in at least one dimension.

10. The bionic organ device according to claim 9, wherein the temperature-sensitive film is suitable for a length change of 1 to 20 μm in at least one dimension.

11. The bionic organ device according to claim 8, wherein the temperature control module further comprises a wire set, and the wire set is connected to the temperature controller and the thermally conductive material layer.
